(19) United States
(12) Patent Application Publication
(10) Pub. No.: US 2024/0148878 A1
(43) Pub. Date: May 9, 2024

(54) THERAPEUTIC POLY-ADP-RIBOSE POLYMER-GCSF CONJUGATES

(71) Applicant: UNIVERSITY OF SOUTHERN CALIFORNIA, Los Angeles, CA (US)

(72) Inventor: Yong ZHANG, Los Angeles, CA (US)

(21) Appl. No.: 18/493,223

(22) Filed: Oct. 24, 2023

Related U.S. Application Data
(60) Provisional application No. 63/380,678, filed on Oct. 24, 2022.

(51) Int. Cl. A61K 47/54; A61K 47/60; A61K 47/64

(52) U.S. Cl. CPC A61K 47/549; A61K 47/60; A61K 47/64

(57) ABSTRACT

A poly ADP-ribose polymerase (PARP)-effector molecule conjugate comprising an automodified PARP having a plurality of poly ADP-ribose (ADPr) polymers on a surface of the automodified PARP, wherein the poly ADPr polymers comprise two or more 3′-azido NAD⁺ moieties, and the effector molecule is conjugated to at least one of the 3′-azido NAD⁺ moieties of the poly ADPr polymers while at least one of the 3′-azido NAD⁺ moieties remain unconjugated.

### RELATED APPLICATIONS

This application claims priority under 35 U.S.C. § 119(e) to U.S. Provisional Patent Application No. 63/380,678 filed Oct. 24, 2022, which application is incorporated herein by reference in its entirety.

### GOVERNMENT SUPPORT

This invention was made with government support under grant no. R35GM137901, awarded by the (NIH/NIGMS) National Institute of General Medical Sciences and grant no. R01EB031830, awarded by the (NIH/NBIB) National Institute of Biomedical Imaging and Bioengineering. The government has certain rights in the invention.

### REFERENCE TO SEQUENCE LISTING

The present application includes a Sequence Listing in electronic format as an xml file titled “530.032US1_SL” which was created on Oct. 23, 2023, and has a size of 7,934 bytes. The contents of xml file 530.032US1_SL are incorporated herein by reference.

### BACKGROUND OF THE INVENTION

Protein poly-ADP-ribosylation (PARylation) occurs through covalent attachments of ADP-ribose moieties from nicotinamide adenine dinucleotide (NAD⁺) onto target proteins. This post-translationally modifying process is catalyzed by endogenous poly(ADP-ribose) polymerases (PARPs) and features formation of linear and branched ADP-ribose polymers with varied sizes and patterns. The resulting heterogeneous ADP-ribose polymers are involved in regulating DNA repair and many other cellular activities. Despite extensive studies of PARylation in physiology and pathophysiology, the potential of the ADP-ribose polymers for therapeutic applications remains untapped. As a natural form of polymer, poly-ADP-ribose (PAR) is characterized by high hydrophilicity and biocompatibility and may therefore serve as promising drug carriers. Accordingly, there is a need for clinically effective targeted compounds and methods of use of such compounds to treat various disorders. The present disclosure satisfies these needs.

### SUMMARY OF THE INVENTION

It was demonstrated that by using a synthetic 3′-azido NAD⁺ molecule with excellent substrate activity for protein PARylation, the derived PAR polymers facilitate efficient conjugation of hydrophobic drugs and monoclonal antibodies for targeted delivery. Considering the polymeric nature of PAR carriers, the therapeutic proteins or other effector molecules conjugated with the PAR may exhibit enhanced efficacy due to increased valency. To test this notion, human granulocyte colony-stimulating factor (GCSF) was chosen as a model ligand for PAR conjugation. GCSF is a 20 kDa neutrophil-mobilizing cytokine. It signals through the GCSF receptor on cell surface. Binding of GCSF leads to receptor dimerization or oligomerization and subsequent activation. Recombinant GCSF in various forms are developed for treatment of neutropenia.

In the present disclosure, human GCSF functionalized with clickable alkynes was conjugated via click chemistry with 3′-azido NAD⁺-derived PAR polymers generated through human PARP1-catalyzed auto-PARylation. The resulting PARylated PARP1-GCSF conjugate displays potent activity in stimulating proliferation of NFS-60 cells. Importantly, mice receiving a single dose of the PARylated PARP1-GCSF conjugate are characterized by prolonged high levels of neutrophil populations, demonstrating therapeutic utility of PAR polymer-protein conjugates in animal models.

In some embodiments, a poly ADP-ribose polymerase (PARP)-effector molecule conjugate comprises an automodified PARP comprising a plurality of poly ADP-ribose (ADPr) polymers, wherein the plurality of poly ADPr polymers comprise a plurality of 3′-azido NAD⁺ moieties; and an effector molecule conjugated to one or more of the plurality of 3′-azido NAD⁺ moieties of the poly ADPr polymers, wherein at least one of the plurality of 3′ -azido NAD⁺ moieties is not conjugated to an effector molecule.

In some embodiments, the two or more 3′ -azido NAD⁺ moieties have a structure according to Formula I:

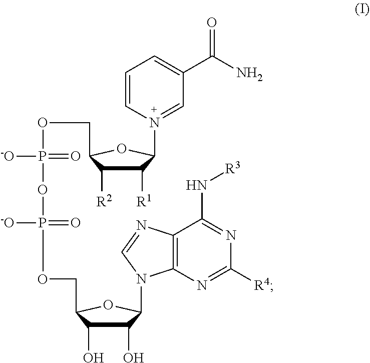

wherein R¹ is OH, R² is N₃, R³ is H, and R⁴ is H. In other embodiments, R¹-R⁴ of Formula I can be defined as shown in Table 1 below.

In some embodiments, the automodified PARP is linked to the effector molecule through an alkyne-derived linking group, a polyethylene glycol linking group, or a linking group that is a polyethylene glycol linking group comprising an alkyne-derived linking moiety. In other embodiments, the modified PARP is linked to the effector molecule through an alkyne-derived linking group, a cyclooctyne-derived linking group, a polyethylene glycol linking group or a combination thereof. In some embodiments, the alkyne-derived linking group is a linking group derived from alkyne-PEGₓ-NHS ester, wherein x is an integer from 2 to about 10. As would be recognized by one of skill in the art, the resulting linking group of any one or more embodiments can be a triazole moiety, for example, a 1,4-disubsituted-1,2,3-triazole (see, for example, FIG. 1).

In some embodiments, the PARP is selected from the group consisting of PARP1, PARP2, PARP5α, and PARP5β. In some embodiments, the PARP is PARP1.

In some embodiments, the effector molecule comprises an interleukin, an interferon, a tumor necrosis factor, a granulocyte colony stimulating factor (GCSF), or a granulocyte-macrophage colony stimulating factor (GM-CSF). In some embodiments, a ratio of the effector molecule conjugated to the automodified PARP is about 5:1 to about 100:1, or about 5:1 to about 20:1, or about 10:1 to about 15:1.

In one embodiment, poly ADP-ribose polymerase (PARP)-granulocyte colony stimulating factor (GCSF) conjugate comprises an automodified PARP1 or PARP2 comprising a plurality of poly ADP-ribose (ADPr) polymers on a surface of the automodified PARP1 or PARP2, wherein the poly ADPr polymers comprise a plurality of 3′ -azido NAD⁺ moieties according to Formula I:

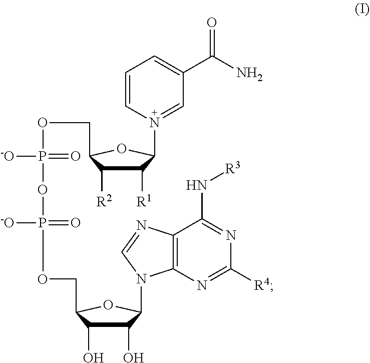

wherein R¹ is OH, R² is N₃, R³ is H, and R⁴ is H; and GCSF conjugated to one or more of the plurality of 3′-azido NAD⁺ moieties of the poly ADPr polymers, wherein a ratio of the GCSF conjugated to the PARP1 or PARP2 is about 5:1 to about 100:1, or about 5:1 to about 50:1, wherein at least one of the plurality of 3′-azido NAD⁺ moieties remain unconjugated, or alternatively, R¹-R⁴ of Formula I are as defined in Table 1 below.

In some embodiments, a method of preparing a poly ADP-ribose polymerase (PARP)-effector molecule conjugate comprises combining a linker and an effector molecule to provide an effector molecule-linker conjugate; combining a PARP and a plurality of an azido substituted dinucleotide to provide an automodified PARP comprising a plurality of poly ADP-ribose (ADPr) groups on a surface of the automodified PARP; and combining the automodified PARP and the effector molecule-linker conjugate under suitable conditions such that the effector molecule-linker conjugate is conjugated to the substituted dinucleotide through click chemistry to form the PARP-effector molecule conjugate.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1 illustrates a schematic of the design and generation of a PARylated PARP1-GCSF conjugate.

FIG. 2A-D illustrates the characterization of the PARylated PARP1-GCSF conjugate. (A) and (B) Purified recombinantly expressed human PARP1 and PARylated PARP1 by 3′-azido NAD⁺ as analyzed by Coomassie stain (A) and immunoblotting (B) using an anti-PAR antibody. (C) and (D) Conjugation of recombinantly expressed human GCSF with PARylated PARP1 by 3 ′-azido NAD⁺ as analyzed by Coomassie stain (C) and immunoblotting (D) using an anti-human GCSF antibody.

FIG. 3 illustrates the PARylated PARP1-GCSF conjugate stimulates proliferation of mouse NFS-60 cells in a dose-dependent manner. Cells were treated with various concentrations of PARylated PARP1, PARylated PARP1-GCSF conjugate, and human GCSF for 72 hours. Cell viabilities were quantified using MTT assays.

FIG. 4A-C illustrates in vivo efficacy of the PARylated PARP1-GCSF conjugate. Single doses of GCSF (1.25 nmol), PARylated PARP1 (2.5 nmol) and PARylated PARP1-GCSF conjugate (0.5 or 2.5 nmol) were administrated by subcutaneous (s.c.) injections into CD1 mice (five per group). Blood was collected and analyzed for neutrophil populations by flow cytometry using fluorophore-labeled anti-mouse CD45, anti-mouse CD11b, and anti-mouse Ly-6G antibodies. (A)-(B) Neutrophil percentages in mouse blood after the single-dose treatments. For (B), 1=PBS; 2=GCSF 1.25 nmol; 3=PARylated PARP1 2.5 nmol; 4=PARylated PARP1-GCSF 0.5 nmol; 5=PARylated PARP1-GCSF 2.5 nmol. (C) Representative flow cytometric analyses of mouse neutrophil populations after treatments with 1) PBS; 2) GCSF 1.25 nmol; 3) PARylated PARP1 2.5 nmol; 4) PARylated PARP1-GCSF 0.5 nmol; and 5) PARylated PARP1-GCSF 2.5 nmol conjugate. ns=not significant, p>0.05; *, p<0.05; **, p<0.01; ***, p<0.001; ****, p<0.0001. Data for the 5 conjugates are listed for days 0, 1, 5, and 11.

FIG. 5 illustrates an SDS-PAGE analysis of purified human PARP1 and human GCSF with Coomassie stain.

FIG. 6 illustrates an SDS-PAGE analysis of purified human GCSF and GCSF-alkyne with Coomassie stain.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely”, “only”, “excluding”, and the like, in connection with any element described herein and/or the recitation of claim elements, for example, for use as a “negative” limitation in a claim.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and sub stituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. An appropriate “effective” amount in any individual case may be determined using techniques, such as a dose escalation study.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

“Poly adenosine diphosphate ribose (ADP) transferase activity” intends the activity of Poly-(ADP-ribose) polymerases (PARPs) that are found mostly in eukaryotes and catalyze the transfer of multiple ADP-ribose molecules to target proteins. As with mono-ADP ribosylation, the source of ADP-ribose is NAD⁺. PARPs use a catalytic triad of His-Tyr-Glu to facilitate binding of NAD⁺ and positioning of the end of the existing poly-ADP ribose chain on the target protein; the Glu facilitates catalysis and formation of a (1->2) O-glycosidic linkage between two ribose molecules. There are several other enzymes that recognize poly-ADP ribose chains, hydrolyze them or form branches.

The term “ADP-ribose unit” refers to a moiety comprising, consisting essentially of, or yet further consisting of an adenosine and a ribose joined through a diphosphate group.

“Adenosine diphosphate ribose (ADP) ribosyltransferase activity” intends the intracellular action of the addition of one or more ADP-ribose moieties to a protein. It is a reversible post-translational modification that is involved in many cellular processes, including cell signaling, DNA repair, gene regulation and apoptosis. Improper ADP-ribosylation has been implicated in some forms of cancer.

“Nicotinamide adenine dinucleotide” (NAD⁺) is also known as diphosphopyridine nucleotide (DPN+) and Coenzyme I, a known coenzyme found in all cells. The structure of NAD⁺ is shown below.

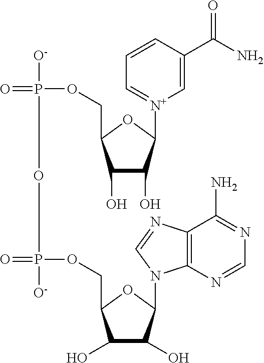

### Nicotinamide Adenine Dinucleotide Analogues

In various embodiments, the nicotinamide adenine dinucleotide (NAD⁺) that acts a donor of ADP-ribose in the automodification and/or PARylation reaction is modified at the 2′-OH (R¹ position), the 3′-OH (R² position) position, the 6-amine position of the purine moiety (R³ position), and/or the 2-position of the purine moiety (R⁴ position) and may include a chemical group that may undergo “click chemistry”. Various embodiments include a modified NAD⁺ analogue having a “click chemistry” group according to Table 1 below.

TABLE 1
Chemical structures of NAD⁺ analogues 1-9.

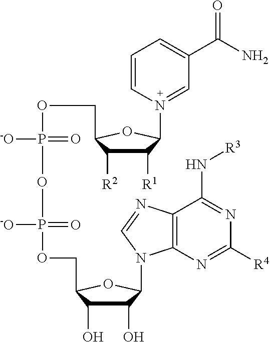

| | | | | |
|---|---|---|---|---|
| 1: | 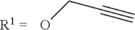 | R² = OH | R³ = H | R⁴ = H |
| 2: | R¹ = OH | 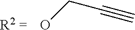 | R³ = H | R⁴ = H |
| 3: | 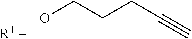 | R² = OH | R³ = H | R⁴ = H |
| 4: | R¹ = OH | 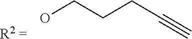 | R³ = H | R⁴ = H |
| 5: | R¹ = N₃ | R² = OH | R³ = H | R⁴ = H |
| 6: | R¹ = OH | R² = N₃ | R³ = H | R⁴ = H |
| 7: | 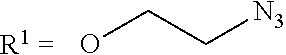 | R² = OH | R³ = H | R⁴ = H |
| 8: | R¹ = OH | R² = OH | 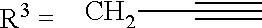 | R⁴ = H |
| 9: | R¹ = OH | R² = OH | R³ = H | 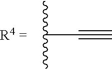 |

Various modified NAD⁺ analogue embodiments include modified NAD⁺ analogues having an alkyne or azido group, such as those shown in Table 1, at any of the R¹-R⁴ positions, in any different combination. For example, any of analogues 1-7 may also be substituted with an alkyne at the R³ or R⁴ positions, and any of analogues 8-9 can be substituted at the R¹ or R² positions with an oxygen-linked alkyne or alkyl azido group, or they can have an azido group in place of a hydroxyl at the R¹ or R² position. In certain preferred embodiments, the NAD⁺ analogue comprises a chemical structure of NAD⁺ analogue 2 (R¹=OH, R²=alkyne, e.g., propargyl), which can be referred to herein as the 3 ′-alkyne compound, or NAD⁺ analogue 6 (R¹=OH, R²=azido), which can be referred to herein as the 3′-azido compound, which compounds have the following structures:

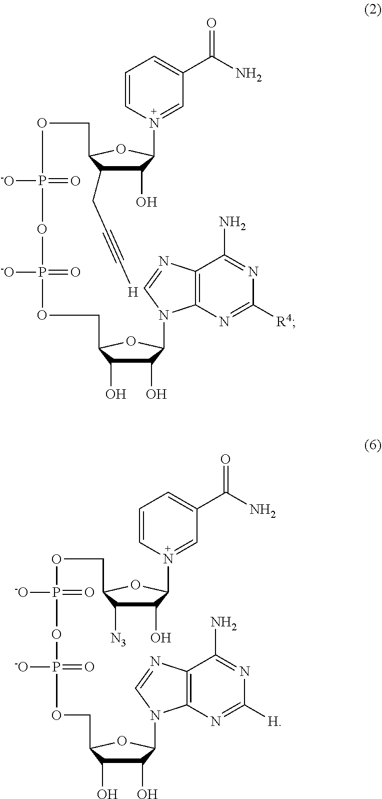

In one embodiment, a modified NAD⁺ analogue having a “click chemistry” group—termed 3′-azido NAD⁺—is the NAD⁺ analogue illustrated below:

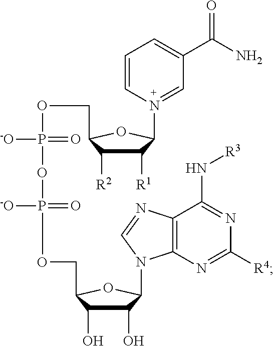

where R¹=OH, R²=N³, R³=H, and R⁴=H. In various embodiments, the ADPr polymers on the surface of the PARP protein comprise a 3 ‘-azido moiety wherein an effector molecule is conjugated to at least one but less than all of the 3’-azido moieties. In some embodiments, the effector molecule is conjugated to a 3′-azido moiety of the ADPr polymers comprising the modified NAD⁺ analogues via a linker moiety.

### PARPs

Poly-ADP-ribose polymerases (PARPs), also known as ADP-ribosyltransferases (ARTs), are emerging as major effectors of NAD⁺-mediated signaling in cells. PARPS are a diverse family of at least 17 mammalian enzymes that catalyze the reversible post-translational modification—known as ADP-ribosylation, or PARylation—involving the transfer of ADP-ribose from NAD⁺ to target proteins. This enzymatic posttranslational modification requires nicotinamide adenine dinucleotide (NAD⁺) as a donor of ADP-ribose. Upon covalent attachments of ADP-riboses to side chains of various types of amino acid residues, PARPs may continue adding ADP-ribose sequentially at ribosyl 2′-OH positions, resulting in linear or branched poly-ADP-ribose (PAR) polymers with up to 300 ADP-ribose units in length. Exemplary PARPS include, but are not limited to, PARP1, PARP2, PARP5α, and PARP5β.

### Effector Molecules

Embodiments of the disclosure may comprise one or more effector molecules conjugated—preferably via click chemistry as described herein—to the plurality of ADPr polymers disposed on the surface of the automodified PARP. Suitable effector molecules include, for example, human growth hormone, growth hormone releasing hormone, growth hormone releasing peptide, interferons (e.g., α, β, γ) and interferon receptors (e.g., interferon-α, -β and -γ, water soluble type I interferon receptor, etc.), granulocyte colony stimulating factor (GCSF), granulocyte-macrophage colony stimulating factor (GM-CSF), glucagon-like peptides (e.g., GLP-1, etc.), G-protein-coupled receptor, interleukins (e.g., interleukin-1, -2, -3, -4, -5, -6, -7, -8, -9, -10, -11, -12, -13, -14, -15, -16, -17, -18, -19, -20, -21, -22, -23, -24, -25, -26, -27, -28, -29, -30, etc.) and interleukin receptors (e.g., IL-1 receptor, IL-4 receptor, etc.), enzymes (e.g., glucocerebrosidase, iduronate-2-sulfatase, alpha-galactosidase-A, agalsidase alpha and beta, alpha-L-iduronidase, butyrylcholinesterase, chitinase, glutamate decarboxylase, imiglucerase, lipase, uricase, platelet-activating factor acetylhydrolase, neutral endopeptidase, myeloperoxidase, etc.), interleukin and cytokine binding proteins (e.g., IL-18 bp, TNF-binding protein, etc.), macrophage activating factor, macrophage peptide, B cell factor, T cell factor, protein A, allergy inhibitor, cell necrosis glycoproteins, immunotoxin, lymphotoxin, tumor necrosis factor (α, β, γ), tumor suppressors, metastasis growth factor, alpha-1 antitrypsin, albumin, alpha-lactalbumin, apolipoprotein-E, erythropoietin, highly glycosylated erythropoietin, angiopoietins, hemoglobin, thrombin, thrombin receptor activating peptide, thrombomodulin, factor VII, factor VIIa, factor VIII, factor IX, factor XIII, plasminogen activating factor, fibrin-binding peptide, urokinase, streptokinase, hirudin, protein C, C-reactive protein, renin inhibitor, collagenase inhibitor, superoxide dismutase, leptin, platelet-derived growth factor, epithelial growth factor, epidermal growth factor, angiostatin, angiotensin, bone growth factor, bone stimulating protein, calcitonin, insulin, atriopeptin, cartilage inducing factor, elcatonin, connective tissue activating factor, tissue factor pathway inhibitor, follicle stimulating hormone, luteinizing hormone, luteinizing hormone releasing hormone, nerve growth factors (e.g., nerve growth factor, ciliary neurotrophic factor, axogenesis factor-1, brain-natriuretic peptide, glial derived neurotrophic factor, netrin, neutrophil inhibitor factor, neurotrophic factor, neuturin, etc.), parathyroid hormone, relaxin, secretin, somatomedin, insulin-like growth factor, adrenocortical hormone, glucagon, cholecystokinin, pancreatic polypeptide, gastrin releasing peptide, corticotropin releasing factor, thyroid stimulating hormone, autotaxin, lactoferrin, myostatin, receptors (e.g., TNFR(P75), TNFR(P55), IL-1 receptor, VEGF receptor, B cell activating factor receptor, etc.), receptor antagonists (e.g., IL1-Ra etc.), cell surface antigens (e.g., CD 2, 3, 4, 5, 7, 11a, 11b, 18, 19, 20, 23, 25, 33, 38, 40, 45, 69, etc.), monoclonal antibodies, polyclonal antibodies, antibody fragments (e.g., scFv, Fab, Fab′, F(ab′)2 and Fd), and virus derived vaccine antigens.

In one specific embodiment, the effector molecule is granulocyte colony stimulating factor (GCSF) (NCBI Reference No. NP_000750.1; NM_000759.4).

In some embodiments, the effector molecule is conjugated to a PARP via the ADPr polymers comprising a substituted dinucleotide in a ratio (effector molecule:PARP) of about 5:1 to about 100:1, about 5:1 to about 50:1, about 5:1 to about 25:1, about 10:1 to about 20:1, or about 15:1.

### Linker Groups

In certain embodiments, the linker may include a functional group or moiety that is capable of undergoing a click chemistry reaction. The click chemistry approach was originally conceived as a method to rapidly generate complex substances by joining small subunits together in a modular fashion. (See, e.g., Evans, R A, 2007, Aust J Chem 60(6):384-95.) Multiple variations of click chemistry reaction are known in the art, such as the Huisgen 1,3-dipolar cycloaddition copper catalyzed reaction (Tornoe et al., 2002, J. Org. Chem. 67:3057-64). Other alternative reaction mechanisms include cycloaddition reactions such as the Diels-Alder, nucleophilic substitution reactions (especially to small, strained rings like epoxy and aziridine compounds), carbonyl chemistry formation of urea compounds and reactions involving carbon-carbon double bonds, such as alkynes in thiol-yne reactions.

The azide alkyne Huisgen cycloaddition reaction uses a copper catalyst in the presence of a reducing agent to catalyze the reaction of a terminal alkyne group attached to a first molecule. In the presence of a second molecule comprising, for example, an azide moiety, the azide reacts with the activated alkyne to form a 1,4-disubstituted 1,2,3-triazole. Advantageously the copper catalyzed reaction occurs at room temperature and is sufficiently specific that purification of the reaction product is often not required (Tornoe et al., 2002, J. Org. Chem 67:3057). Advantageously, the azide and alkyne functional groups are largely inert towards biomolecules in aqueous medium, thus permitting the reaction to occur in complex solutions. The resultant triazole is chemically stable and may not be subject to enzymatic cleavage, making the click chemistry product highly stable in biological systems. And although the copper catalyst is toxic to living cells, the copper-based click chemistry reaction may be used in vitro for immunoconjugate formation.

A copper-free click reaction also has been proposed for covalent modification of biomolecules. (See, e.g., Agard et al., J. Am. Chem. Soc. 2004, 126, 46, 15046-15047). In this system, the copper-free reaction uses ring strain in place of the copper catalyst to promote a [3+2] azide-alkyne cycloaddition reaction. For example, cyclooctyne is an 8-carbon ring structure comprising an internal alkyne bond. The closed ring structure induces a substantial bond angle deformation of the acetylene, which is highly reactive with azide groups to form a triazole. Thus, cyclooctyne derivatives may be used for copper-free click reactions.

Another type of copper-free click reaction (Ning et al, Angew Chem Int Ed Engl. 2010 Apr. 12; 49(17): 3065-3068.) is based upon strain-promoted alkyne-nitrone cycloaddition. To address the slow rate of the original cyclooctyne reaction, electron-withdrawing groups are attached adjacent to the triple bond. Examples of such substituted cyclooctynes include difluorinated cyclooctynes, 4-dibenzocyclooctynol and azacyclooctyne. An alternative copper-free reaction involved strain-promoted alkyne-nitrone cycloaddition to give N-alkylated isoxazolines. The reaction was reported to have exceptionally fast reaction kinetics and was used in a one-pot three-step protocol for site-specific modification of peptides and proteins. Nitrones were prepared by the condensation of appropriate aldehydes with N-methylhydroxylamine and the cycloaddition reaction took place in a mixture of acetonitrile and water. These and other known click chemistry reactions may be used to attach carrier moieties to antibodies in vitro.

In some embodiments of the click chemistry reaction, the reactive group comprises an alkyne that is capable of undergoing a 1,3-cycloaddition reaction with an azide. Such suitable reactive groups include, but are not limited to, strained alkynes, e.g., those suitable for strain-promoted alkyne-azide cycloadditions (SPAAC), cycloalkynes, e.g., cyclooctynes, benzannulated alkynes, and alkynes capable of undergoing 1,3-cycloaddition reactions with azides in the absence of copper catalysts. Suitable alkynes also include, but are not limited to, substituted alkynes, e.g., fluorinated alkynes, aza-cycloalkynes, bicyclo[6.1.0]nonyne (BCN), and derivatives thereof. Linker-effector molecules comprising such reactive groups are useful for conjugating molecules that have been functionalized with azido groups.

In various embodiments, the linker includes a reaction group capable on undergoing a click chemistry reaction comprising an azide, alkyne, dibenzocyclooctyne, trans-cyclooctene, tetrazine, and bicyclo[6.1.0]nonyne. In one preferred embodiment, the linker is endo-BCN-PEG4-NHS ester. Commercial endo-BCN-PEG4-NHS ester linkers may be used to conjugate the BCN moieties rapidly on to the primary amines of an effector molecule. BCN groups are able to react with azide groups on an automodified PARP (e.g., PARP1) through copper free click chemistry, bridging the effector molecule together with automodified PARP (Leunissen et al., Chembioche, 2014 Jul. 7; 15(10): 1446-51).

In certain embodiments, the solubility of the effector molecule may be enhanced by placing a defined polyethyleneglycol (PEG) moiety (i.e., a PEG containing a defined number of monomeric units) between the effector molecule and the ADPr polymer, wherein the defined PEG is a low molecular weight PEG, preferably containing 1-30 monomeric units, more preferably containing 1-12 monomeric units.

In some embodiments, the linker group is alkyne-PEG4-NHS ester linker having the structure:

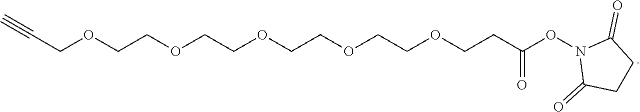 [SMILES: C#CCOCCOCCOCCOCCOCCC(=O)ON1C(=O)CCC1=O]

Importantly, while it is known in the art that “click chemistry” is an efficient chemical reaction that may be used to conjugate various moieties to a substrate, it is also well known in the art that even click chemistry reactions do not proceed to absolute 100% completion, regardless of the amount and type of reactants used. In line with this principle, the ADPr polymers will comprise at least one substituted dinucleotide (e.g., 3′-azido NAD⁺ moieties) with an unreacted azide moiety (i.e., that have not reacted with an effector molecule) in the final polymer structure.

In some embodiments, a conjugate comprises an automodified PARP comprising a plurality of poly ADP-ribose (ADPr) polymers on a surface of the automodified PARP, wherein the plurality of poly ADPr polymers comprise a plurality of 3′-azido NAD⁺ moieties; and an effector molecule conjugated to one or more of the 3′-azido NAD⁺ moieties of the poly ADPr polymers, wherein at least one of the plurality of 3′-azido NAD⁺ moieties is not conjugated to an effector molecule (i.e., remains unconjugated).

In some embodiments, the 3′-azido NAD⁺ moieties have a structure according to Formula I:

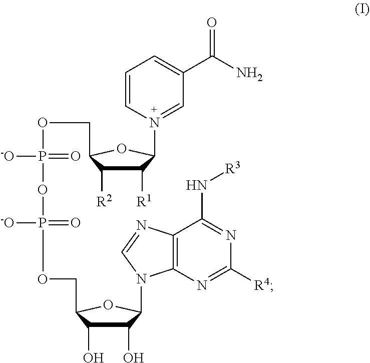

wherein R¹ is OH, R² is N₃, R³ is H, and R⁴ is H.

In some embodiments, a poly ADP-ribose polymerase (PARP)-granulocyte colony stimulating factor (GCSF) conjugate comprises: an automodified PARP1 or PARP2 comprising a plurality of poly ADP-ribose (ADPr) polymers on a surface of the automodified PARP1 or PARP2, wherein the poly ADPr polymers comprise a plurality of 3′-azido NAD⁺ moieties according to Formula I:

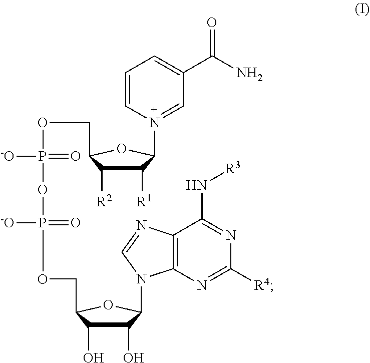

wherein R¹ is OH, R² is N₃, R³ is H, and R⁴ is H; and GCSF conjugated to one or more of the plurality of 3′-azido NAD⁺ moieties of the poly ADPr polymers, wherein a ratio of the GCSF conjugated to the PARP1 or PARP2 is about 5:1 to about 100:1, or about 5:1 to about 50:1, wherein at least one of plurality of 3′-azido NAD⁺ moieties remains unconjugated to another moiety.

In some embodiments, a method of preparing a poly ADP-ribose polymerase (PARP)-effector molecule conjugate comprises combining a linker and an effector molecule to provide an effector molecule-linker conjugate; combining a PARP and a plurality of an azido substituted dinucleotide to provide an automodified PARP comprising a plurality of poly ADP-ribose (ADPr) groups on a surface of the automodified PARP; and combining the automodified PARP and the effector molecule-linker conjugate under suitable conditions such that the effector molecule-linker conjugate is conjugated to the substituted dinucleotide through click chemistry to form the PARP-effector molecule conjugate.

The disclosure also provides for a composition comprising a conjugate as described herein and a pharmaceutically acceptable carrier.

In some embodiments, a method of stimulating neutrophil cell proliferation comprises contacting a neutrophil cell with an effective amount of any one of the conjugates described herein, thereby causing proliferation of the neutrophil cell. In some embodiments, neutrophil cell proliferation may be stimulated in response to neutropenia induced by chemotherapy or other drug treatments. In some embodiments, neutrophil cell proliferation may be stimulated in hematopoietic stem cell transplantation.

In some embodiments, an amount of conjugate used to stimulate neutrophil cell growth in either in vitro or in vivo conditions comprises about 0.1 nM to about 10 nM, about 0.5 nM to about 5 nM, about 1 nM to about 3 nM, or about 2.5 nM.

### Pharmaceutical Formulations

The compounds comprising the conjugates described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds comprising the conjugates described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds comprising the conjugates described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compounds (i.e., the PARP-effector molecule conjugate) may be administered intravenously or intraperitoneally by infusion or injection. Solutions of the active compound or its salts can be prepared in water, optionally mixed with a nontoxic surfactant.

Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Useful dosages of the compositions described herein can be determined by comparing their in vitro activity, and in vivo activity in animal models. Methods for the extrapolation of effective dosages in mice, and other animals, to humans are known to the art; for example, see U.S. Pat. No. 4,938,949. The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compounds comprising the conjugates described herein are conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound or composition as described herein formulated in such a unit dosage form.

The compounds comprising the conjugates described herein can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Poly-ADP-Ribose Polymer-GCSF Conjugates

Poly-ADP-ribose (PAR) is a naturally occurring form of polymer synthesized through enzymatic reactions catalyzed by poly(ADP-ribose) polymerases (PARPs). It is known for regulating various important cellular signaling pathways and processes. As a water soluble and biocompatible type of polymer, PAR. may hold promise for safe and efficient delivery of therapeutics. To explore the therapeutic potential of PAR polymers, PAR polymers conjugated with human granulocyte colony-stimulating factor (GCSE) protein were generated by harnessing human PARP1-catalyzed auto-poly-ADP-ribosylation and a clickable analogue of nicotinamide adenine dinucleotide (NAD⁺). The resulting PAR polymer-based conjugate with multivalent GCSE ligands exhibits a potent cell proliferative activity. Notably, mice treated with a single dose of the PAR polymer-GCSF conjugate show sustained high levels of neutrophil in blood for 11 days, demonstrating excellent in vivo efficacy. Functionalized PAR polymers may provide new scaffolds for conjugating with therapeutic proteins or peptides toward improved pharmacological activities.

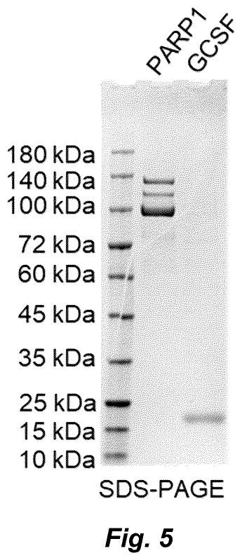

PAR polymers consist of polymeric ADP-ribose in linear and branched formats. Covalent attachments of therapeutic protein ligands to these polymerized ADP-ribose units through orthogonal conjugation are expected to afford PAR polymer-based conjugates with multivalent ligands. Upon engagement with cell surface receptors, such PAR-ligand conjugates may facilitate receptor clustering, consequently promoting activation of receptor-mediated signaling pathways. To this end, full-length human PARP1 was first expressed and purified from Escherichia coli using a previously established bacterial expression vector and protocol (FIG. 5). PARP1 possesses strong catalytic activity for protein PARylation and catalyzes robust auto-PARylation. Human GCSF was then recombinantly expressed in mammalian Expi293F cells through transient transfection and purified to homogeneity via single-step affinity chromatography (FIG. 5).

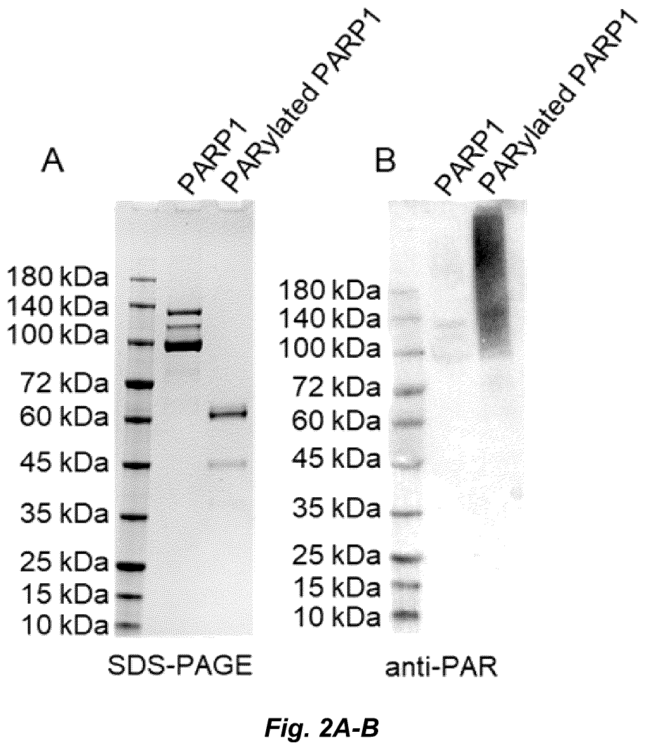

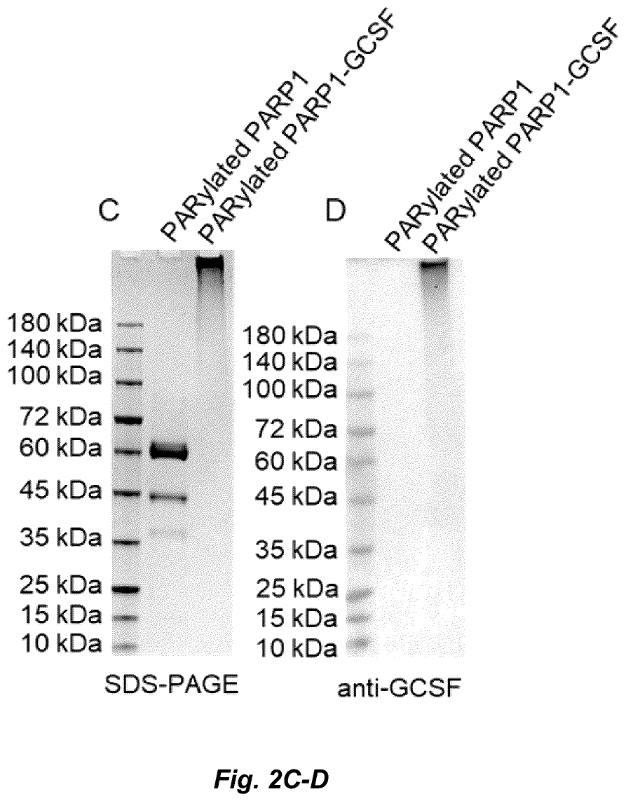

Auto-PARylation of human PARP1 was performed using the synthetic 3′-azido NAD⁺ under previously optimized conditions for producing PARylated PARP1 with clickable ADP-ribose groups. Coomassie-stained SDS-PAGE gels revealed formation of cleaved PARP1 fragments in a range of 35-65 kDa during auto-PARylation (FIG. 2A), likely due to high sensitivity of PARP1 to various proteases. Although the signals of heterogeneous auto-PARylated PARP1 were below the detection threshold of Coomassie stain, generation of 3′-azido NAD⁺-derived PAR polymers was verified by immunoblotting using an anti-PAR monoclonal antibody (FIG. 2B). The anti-PAR immunoblots revealed smeared bands in regions above 100 kDa for PARylated PARP1 modified by 3′-azido NAD⁺, but little or no signals for PARP1, supporting formation of 3′-azido NAD⁺-derived PAR polymers.

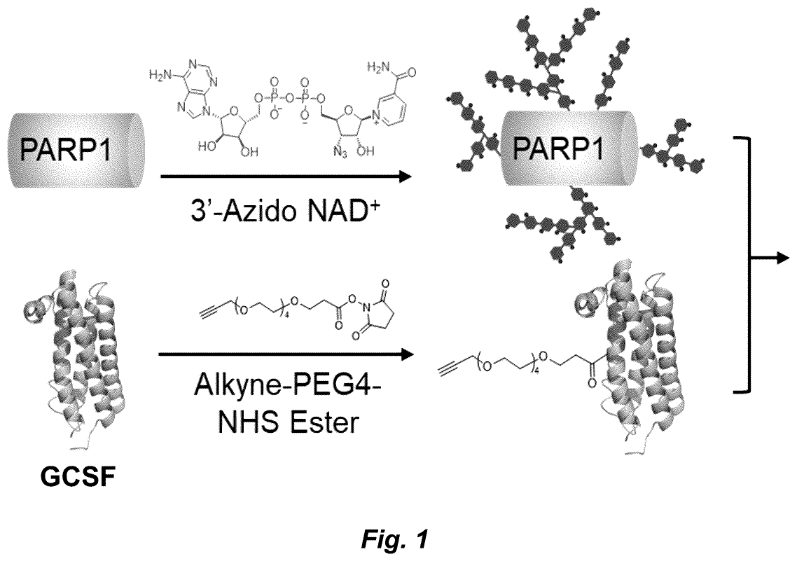

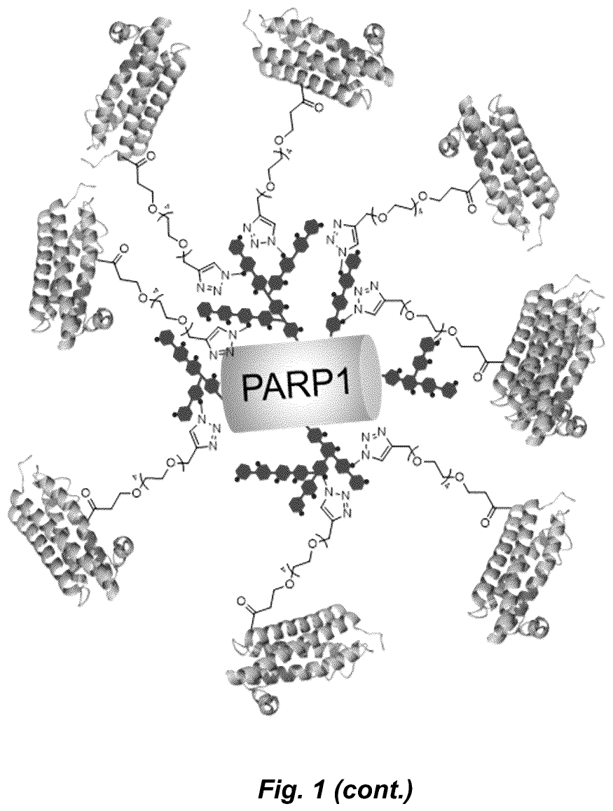

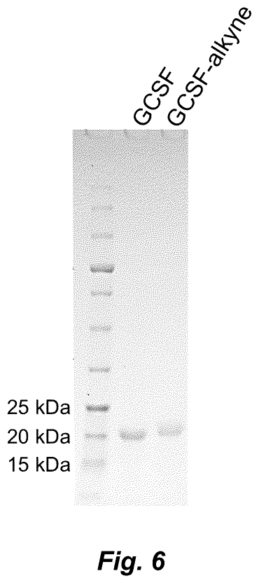

The auto-modified PARP1 by 3′-azido NAD⁺ was then incubated with terminal alkyne-functionalized GCSF (FIG. 6) in copper(I)-catalyzed azide-alkyne cycloaddition (CuAAC) reactions to synthesize the PARylated. PARP1-GCSF conjugate (FIG. 1). Following purification by size-exclusion chromatography, the resulting PARylated PARP1-GCSF conjugate was analyzed by Coomassie-stained SDS-TAGE gels and immunoblotting using an anti-human GCSF monoclonal antibody (FIG. 2C, D). SDS-PAGE analysis by Coomassie stain revealed the generated PARylated PARP1-GCSF conjugate with molecular weights enriched above 180 kDa. The anti-GCSF immunoblots showed that in comparison to PARylated PARP1 lacking signals, the PARylated PARP1-GCSF conjugate is characterized by a smearing pattern mainly over 180 kDa. These results indicate successful conjugation of GCSF to auto-PARylated PARP1 via click chemistry. The amount of GCSF in the PARylated PARP1-GCSF conjugate was then quantified by ELISA (Table 3). On the basis of free GCSF standards, it was calculated that the PARylated PARP1-GCSF conjugate carries 14.0±0.5 GCSF protein molecules on average.

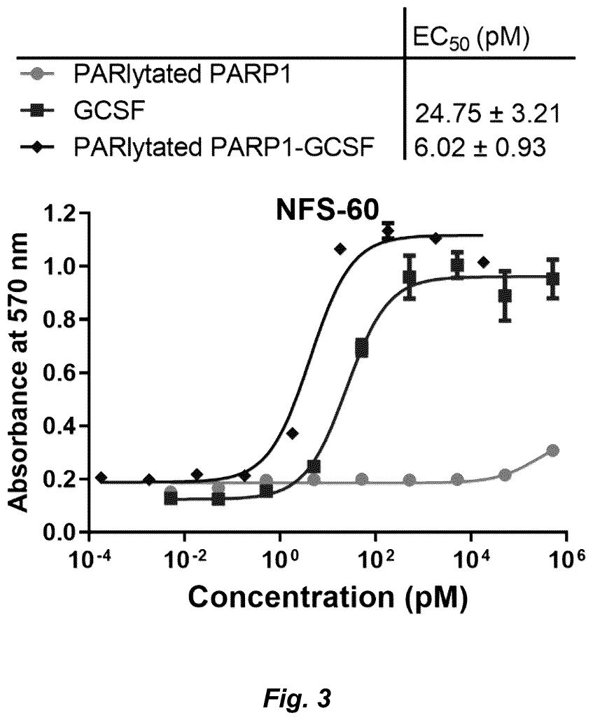

Next, the proliferative activities of GCSF and PARylated PARP1-GCSF conjugate were evaluated using mouse NES-60 cells that are responsive to GCSF stimulation. In vitro cell proliferation assays indicated that recombinant human GCSF shows a dose-dependent stimulatory activity with an EC₅₀ of 24.75±3.21 pM, whereas the PARylated PARP1-GCSF conjugate exhibits approximately 4-fold increased potency (EC₅₀ of 6.02±0.93 pM) (FIG. 3). As a control, PARylated PARP1 displays no proliferative activity under the same conditions. These results demonstrate that conjugation of GCSF cytokine to PARylated PARP1 results in a polymeric conjugate with enhanced biological activity. In comparison with free GCSF, multivalent GCSF ligands on the PARylated PARP1-based conjugate could possibly facilitate lateral stabilization and clustering of cell-surface GCSF receptors, augmenting their signaling activities.

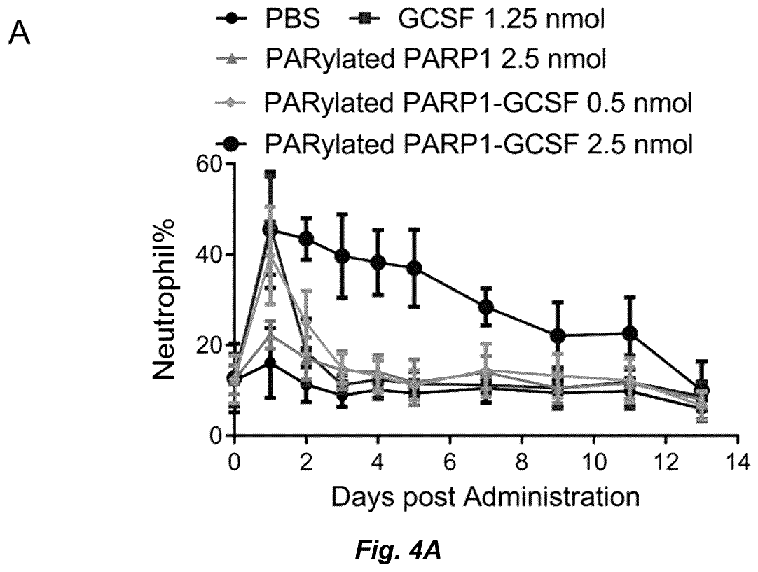

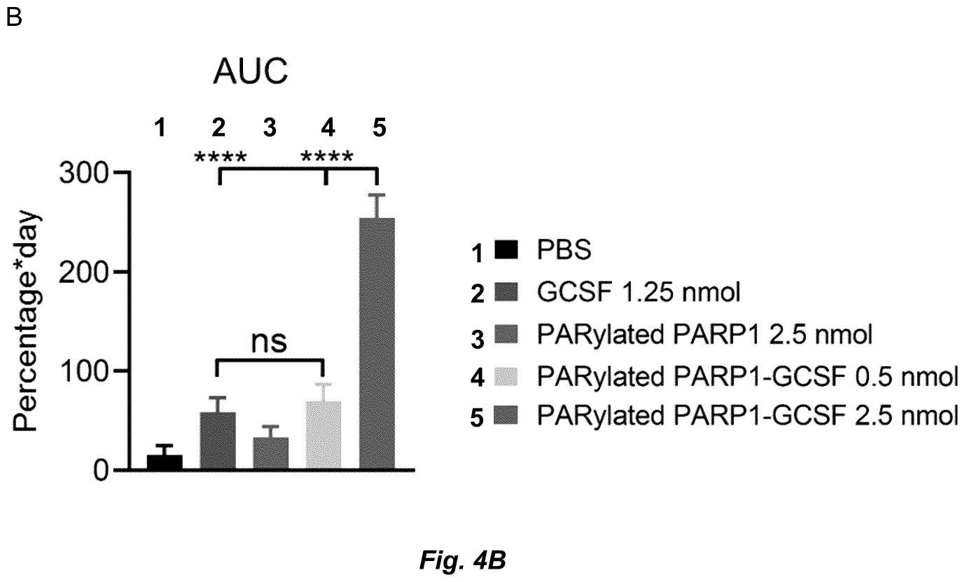

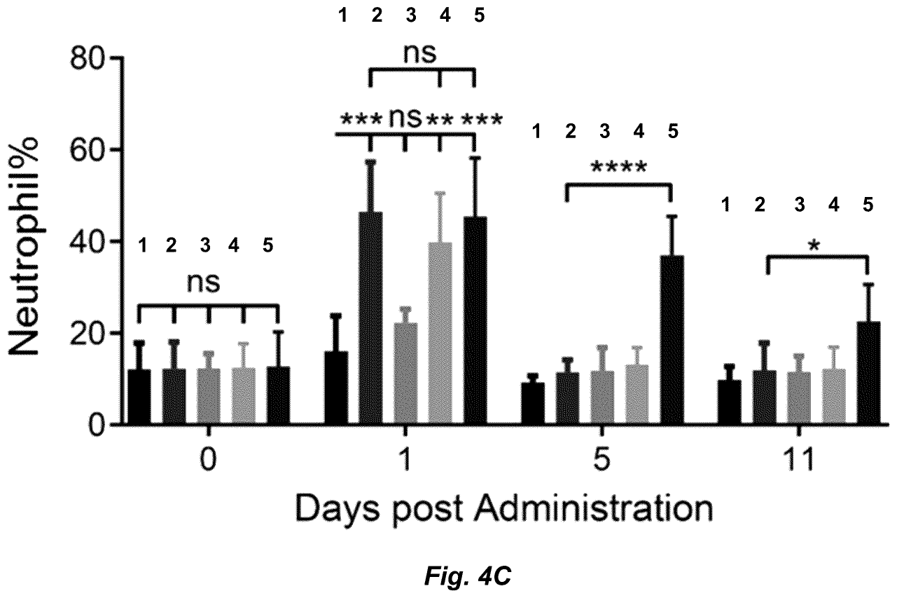

In vivo efficacies of GCSE and PARylated PARP1-GC SF were then examined in mice. Following single-dose treatments, mouse neutrophil populations in blood were analyzed by flow cytometry in next 13 days (FIG. 4). Little or no changes in neutrophil percentage were observed for mice treated with PBS vehicle or PARylated PARP1. In comparison, mice administered with a single dose of GCSF or PARylated PARP1-GCSF conjugate were characterized by sharp increases (approximately from 10% to 50%) of neutrophil percentages within 24 hours. The PARylated PARP1-GCSF conjugate at a dose of 0.5 nmol displays a neutrophil-stimulating activity comparable to that of 1.25 nmol GCSF. Neutrophil levels for both animal groups returned to normal in five days. Notably, sustained elevated levels of neutrophil were seen in first 11 days for mice receiving 2.5 nmol PARylated PARP1-GCSF conjugate. These results support great in vivo activity for the PARylated PARP1-GCSF conjugate, suggesting enhanced potency for the GCSF ligands resulted from conjugation to PAR polymers.

This study demonstrates generation of a PAR polymer-GCSF conjugate by utilizing auto-modified PARP1 and 3′-azido NAD⁺ as well as its marked biological activities. The functionalized PAR polymers carrying clickable groups enable rapid and bioorthogonal conjugation, resulting in the synthesized conjugate with multivalent GCSF ligands which can possibly promote oligomerization of their cognate receptors for upregulated signaling. PAR polymers provide an innovative approach for producing protein conjugates with increased valency. In addition to 3′-azido NAD⁺-derived PAR polymers, functionalized PAR polymers with one or more distinct groups for chemical conjugation could be synthesized and explored. The number of GCSF protein molecules within each conjugate may need optimization for improved activities. Future studies include comparative analysis of pharmacokinetics, biodistribution, efficacy, and toxicity of GCSF, engineered forms of GCSF, and the PAR polymer-GCSF conjugate as well as development of PAR polymer conjugates with different therapeutic peptides or proteins.

A PARylated PARP1-GCSF conjugate was generated by exploiting azido-functionalized PAR polymers formed from PARP1-catalyzed auto-PARylation. This PAR polymer-based conjugate displays excellent in vitro and in vivo stimulatory activities, providing a new polymer scaffold for potential therapeutic development.

TABLE 2
Primer sequences used for molecular cloning of full-length human PARP1 and GCSF.

| DNA fragment | sequence |
|---|---|
| for cloning | Primer |
| | Forward: 5′-TGGTGCTCGAGCCACAGGGAGGTCTTAAAATTGAATT TCAGT-3′ |
| | (SEQ ID NO: 3) |
| | Reverse: 5′- |
| | CCCTCTAGAAATAATTTTGTTTAACTTTAAGAAGGAGATATACCATGGCGGAGTCTTC |
| | GGATAAGC-3′ (SEQ ID NO: 4) |
| GCSF | Forward: 5′-GAATTCGACACCACTGGGACCTGCAAGTTC-3′ (SEQ ID NO: 5) |
| | Reverse: 5′-CCAGCTAGCACCTATCAATGGTGGT-3′ (SEQ ID NO: 6) |

TABLE 3
Representative ELISA data for quantification
of GCSF on PARylated PARP1-GCSF conjugate.

| | Fluorescence Intensity | | | |
|---|---|---|---|---|
| | | | PARylated | |
| Concentration | GCSF | | PARP1-GCSF conjugate | |
| (ng/mL) | Replicate #1 | Replicate #2 | Replicate #1 | Replicate #2 |
| 500 | 75706 | 76463 | 64501 | 64887 |
| 250 | 62198 | 60717 | 49437 | 49194 |
| 125 | 42333 | 42733 | 32115 | 44700 |
| 62.5 | 29379 | 29792 | 22842 | 20741 |
| 31.25 | 19874 | 20742 | 15884 | 15924 |

### Example 2. Materials and Methods

Unless otherwise specified, reagents were purchased from common commercial sources and used as received without further purification. Roswell Park Memorial Institute (RPMI) 1640 medium was purchased from Corning Inc. (NY). Opti-modified Eagle's medium (Opti-MEM) and fetal bovine serum (FBS) were purchased from Thermo Fisher Scientific (Waltham, MA). BalanCD HEK293 medium and L-glutamine solution (200 mM) were purchased from FUJIFILM Irvine Scientific (Irvine, CA).

Cell lines. Mouse NFS-60 cells were grown in RPMI 1640 medium containing 10% FBS, 0.05 mM 2-mercaptoethanol and 62 ng/mL human recombinant macrophage colony stimulating factor (M-CSF). Expi293F cells were purchased from Thermo Fisher Scientific and cultured in Expi293F expression medium with shaking at a speed of 125 rpm at 37° C. and 8% CO₂.

Animals. Female CD-1 IGS mice (22-24 g) were purchased from Charles River Laboratories (Wilmington, MA). All animal studies were performed under a protocol approved by the Institutional Animal Care and Use Committee of the University of Southern California.

Chemical synthesis of 3′-azido NAD⁺. The 3′-azido NAD⁺ was prepared according to Zhang et al., Nature communications 2019, 10 (1), 4196, and U.S. Pat. Pub. No. 2022/0088112, which are incorporated herein by reference in their entirety).

Molecular cloning. pET-28a (+) vector encoding the full-length human PARP1 with a C-terminal His6 tag was generated according to Zhang et al., Nature communications 2019, 10 (1), 4196. Synthetic genes encoding human GCSF-His₆ were purchased from Integrated DNA Technologies, Inc. (Skokie, IL). The GCSF-His₆ DNA fragment was amplified using primers listed in Table 2 and then ligated in-frame using T4 DNA ligase between the EcoRI and NheI restriction enzyme sites in a pFuse vector for the generation of a mammalian expression construct of pFuse-hGCSF-His₆ that was verified by DNA sequencing.

Protein expression and purification. The bacterial expression and purification of human PARP1 were carried out as described in Shi et al., Chemical Science 2020, 11 (34), 9303-9308. The purified protein was further passed through an acrodisc unit with mustang E membrane (Pall Corporation, Port Washington, NY) by following the manufacturer's instructions. The final endotoxin levels (<0.5 EU mg⁻¹ mL⁻¹) were measured using Pierce LAL chromogenic endotoxin quantitation kits (Thermo Fisher Scientific). Purified PARP1 was analyzed by SDS-PAGE, flash frozen using liquid nitrogen, and stored at −80° C.

Recombinant human GCSF was expressed through transient transfection into Expi293F cells using the polyethylenimine max (PEI-MAX) transfection regent. Culture media of Expi293F cells transfected with the expression construct were collected at days 3 and 6 post-transfection and centrifuged at 4,000×g for 30 min. The supernatant was dialyzed in PBS buffer for overnight and another 6 hours in PBS at 4° C. and then loaded on a gravity flow column packed with 2 mL of Ni-NTA agarose resin (Thermo Fisher Scientific), followed by washing with PBS containing 20 mM imidazole and eluting with PBS containing 400 mM imidazole. GCSF protein was then dialyzed in PBS buffer for overnight and another 6 hours in PBS at 4° C. and concentrated using a 10 kDa-cutoff amicon centrifugal concentrator. Purified human GCSF was analyzed by SDS-PAGE and stored at −80° C.

GCSF NHS-alkyne linker conjugation. A 20-fold molar excess of alkyne-PEG4-NHS ester linker (Click Chemistry Tools, Scottsdale, AZ; dissolved in 100% DMSO) was added into GCSF protein in PBS. The solution was mixed gently and allowed to react for 2 hours at room temperature. Unreacted linkers were removed through buffer exchange using a 10 kDa-cutoff amicon centrifugal concentrator in PBS. Resulting GCSF-alkyne was analyzed by SDS-PAGE and stored at −80° C.

PARP automodification. Purified PARP (e.g., PARP1) (3 μM) was incubated with 150 μM of 3′-azido NAD⁺ or NAD⁺ in a reaction buffer containing 30 mM HEPES (pH 8.0), 5 mM MgCl₂, 5 mM CaCl₂, 250 mM NaCl, 1 mM DTT and 100 ng μL⁻¹ activated DNA (Sigma-Aldrich, St. Louis, MO) at 30° C. for 12 hours. The reaction mixtures were then buffer exchanged to PBS using a 30 kDa-cutoff amicon centrifugal concentrator.

Conjugation of GCSF-alkyne with PARylated PARP1. GCSF-alkyne was conjugated onto PARylated PARP1 with a molar ratio of 20:1 through copper(I)-catalyzed azide alkyne cycloaddition (CuAAC). Click reactions were performed for 3 days at room temperature, which contained 3 μM of PARylated PARP1, 60 μM GCSF-Alkyne, 2 mM tris-hydroxypropyltriazolylmethylamine (THPTA), 1 mM CuSO₄, and 10 mM sodium ascorbate in PBS. PARylated PARP1-GCSF conjugate was purified through size-exclusion chromatography using a Superdex 200 Increase 10/300 GL column (GE Healthcare, Princeton, NJ) and eluted with PBS. The first peak eluted was collected and concentrated using amicon centrifugal concentrators with 30 kDa-cutoff. Purified PARylated PARP1-GCSF conjugate was examined by SDS-PAGE.

Quantification of GCSF on PARylated PARP1-GCSF conjugate. The GCSF concentrations of PARylated PARP1-GCSF conjugate were measured by ELISA with recombinant GC SF as standards. Ninety-six-well ELISA plates were coated with an anti-GCSF monoclonal antibody (clone: BVD13-3A5, BioLegend, CA) overnight at 4° C. Non-bound antibodies were washed away with PBST (PBS with 0.05% Tween-20) three times. The wells were then blocked with PBS containing 1% bovine serum albumin (BSA) for 2 hours, followed by washing with PBST. Various concentrations of GCSF and PARylated PARP1-GCSF conjugate were added and incubated for 2 hours, followed by washing with PB ST. An anti-GCSF antibody-HRP conjugate (clone 3D1, Santa Cruz Biotechnology) was subsequently added for 2-hour incubation, followed by washing with PBST. QuantaBlu fluorogenic peroxidase substrate (Thermo Fisher Scientific) was then added and the fluorescence signals were measured using a BioTek Synergy H1 Hybrid Multi-Mode Microplate reader (BioTek, VT, USA). The assays were independently repeated four times with at least two replicates for each. The ratios of GCSF protein within the PARylated PARP1-GCSF conjugate were determined on the basis of fluorescence intensities of PARylated PARP1-GCSF conjugate and GCSF standard at the same concentrations for calculating the average number of GCSF protein molecules per conjugate.

Immunoblot analysis. PARylated PARP1 and purified PARylated PARP1-GCSF conjugate (2 μg of protein) were boiled with 10 mM DTT in NuPAGE LDS sample buffer (Thermo Fisher Scientific) at 98° C. for 5 minutes. Samples were then run on 4-20% ExpressPlus-PAGE gels (GenScript, Piscataway, NJ), transferred to immun-blot PVDF membranes (Bio-Rad Laboratories, Inc.). The membranes were subsequently blocked with 5% BSA in PBS with 0.1% Tween-20 for 1 hour at room temperature, followed by incubation with appropriate primary antibodies (anti-poly-ADP-ribose (PAR) monoclonal antibody (clone: 10H) from Santa Cruz Biotechnology, and anti-GCSF (clone: BVD13-3A5) from BioLegend) and secondary antibodies (anti-mouse IgG-HRP (catalog: 62-6520), and anti-rat IgG-HRP (catalog #31470) from Thermo Fisher Scientific). The immunoblots were developed by additions of supersignal west pico PLUS chemiluminescent substrate (Thermo Fisher Scientific) and imaged with a ChemiDoc Touch Imaging System (Bio-Rad Laboratories, Inc.).

In vitro proliferation assays. Mouse NFS-60 cells were cultured in RPMI 1640 medium containing 10% FBS, 0.05 mM 2-mercapoethanol and 62 ng/ml human recombinant M-CSF. To examine the proliferative activity of PARylated PARP1-GCSF conjugate, cells were washed three times with DPBS buffer, resuspended in RPMI-1640 medium with 10% FBS and 0.05 mM 2-mercapoethanol, plated in 96-well plates (1.5×10⁴ cells per well) with various concentrations of GCSF, PARylated PARP1, or PARylated PARP1-hG-CSF conjugate, and incubated for 72 hours at 37° C. with 5% CO₂. Cells were then treated with 0.5 mg mL⁻¹ 3-(4,5-dimethylthiazol-2-yl)-2,5-diphenyltetrazolium bromide (MTT) solution for 3 hours at 37° C., followed by additions of 100 μL of lysis buffer (20% SDS in 50% dimethylformamide, 0.5% (v:v) 80% acetic acid, 0.4% (v:v) 1 N HCl, pH 4.7) and incubated for 4 hours at 37° C. Fluorescence intensity measured at 595 nm is proportional to cell viability.

In vivo efficacy of the PARylated PARP1-GCSF conjugate. Single doses of GCSF (1.25 nmol), PARylated PARP1 (2.5 nmol), or PARylated PARP1-GCSF conjugate (0.5 or 2.5 nmol) were administrated by subcutaneous (s.c.) injections into CD1 mice (5 per group). Blood was collected at day 0 through day 13 and analyzed by flow cytometry to measure percentages of neutrophil populations in white blood cells using pacific blue anti-mouse CD45 (clone: 30-F11, BioLegend), APC anti-mouse CD11b (clone: M1/70, eBioscence, Carlsbad, CA), and FITC anti-mouse Ly-6G antibodies (clone: 1A8, BioLegend).

Statistical analysis. Two-tailed unpaired t tests were performed for comparison between two groups. One-way ANOVA with Tukey post-hoc tests were carried out for comparing multiple groups. A p<0.05 was considered statistically significant. Significance of finding was defined as: ns=not significant, p>0.05; *, p<0.05; **, p<0.01; ***, p<0.001; ****, p<0.0001. Data are shown as mean±SD. All statistical analyses were calculated using GraphPad Prism (GraphPad Software, La Jolla, CA).

### Example 3. Granulocyte Colony-Stimulating Factor Sequences

Human GCSF protein sequence:
(SEQ ID NO: 1)
MAGPATQSPMKLMALQLLLWHSALWTVQEATPLGPASSLPQSFLLKCLE
QVRKIQGDGAALQEKLVSECATYKLCHPEELVLLGHSLGIPWAPLSSCP
SQALQLAGCLSQLHSGLFLYQGLLQALEGISPELGPTLDTLQLDVADFA
TTIWQQMEELGMAPALQPTQGAMPAFASAFQRRAGGVLVASHLQSFLEV
SYRVLRHLAQP (UniProt No. P09919)
Human GCSF DNA sequence:
(SEQ ID NO: 2)
AGCCCGGAGCCTGCAGCCCAGCCCCACCCAGACCCATGGCTGGACCTGC
CACCCAGAGCCCCATGAAGCTGATGGCCCTGCAGCTGCTGCTGTGGCAC
AGTGCACTCTGGACAGTGCAGGAAGCCACCCCCCTGGGCCCTGCCAGCT
CCCTGCCCCAGAGCTTCCTGCTCAAGTGCTTAGAGCAAGTGAGGAAGAT
CCAGGGCGATGGCGCAGCGCTCCAGGAGAAGCTGGTGAGTGAGTGTGCC
ACCTACAAGCTGTGCCACCCCGAGGAGCTGGTGCTGCTCGGACACTCTC
TGGGCATCCCCTGGGCTCCCCTGAGCAGCTGCCCCAGCCAGGCCCTGCA
GCTGGCAGGCTGCTTGAGCCAACTCCATAGCGGCCTTTTCCTCTACCAG
GGGCTCCTGCAGGCCCTGGAAGGGATCTCCCCCGAGTTGGGTCCCACCT
TGGACACACTGCAGCTGGACGTCGCCGACTTTGCCACCACCATCTGGCA
GCAGATGGAAGAACTGGGAATGGCCCCTGCCCTGCAGCCCACCCAGGGT
GCCATGCCGGCCTTCGCCTCTGCTTTCCAGCGCCGGGCAGGAGGGGTCC
TGGTTGCCTCCCATCTGCAGAGCTTCCTGGAGGTGTCGTACCGCGTTCT
ACGCCACCTTGCCCAGCCCTGAGCCAAGCCCTCCCCATCCCATGTATTT
ATCTCTATTTAATATTTATGTCTATTTAAGCCTCATATTTAAAGACAGG
GAAGAGCAGAACGGAGCCCCAGGCCTCTGTGTCCTTCCCTGCATTTCTG
AGTTTCATTCTCCTGCCTGTAGCAGTGAGAAAAAGCTCCTGTCCTCCCA
TCCCCTGGACTGGGAGGTAGATAGGTAAATACCAAGTATTTATTACTAT
GACTGCTCCCCAGCCCTGGCTCTGCAATGGGCACTGGGATGAGCCGCTG
TGAGCCCCTGGTCCTGAGGGTCCCCACCTGGGACCCTTGAGAGTATCAG
GTCTCCCACGTGGGAGACAAGAAATCCCTGTTTAATATTTAAACAGCAG
TGTTCCCCATCTGGGTCCTTGCACCCCTCACTCTGGCCTCAGCCGACTG
CACAGCGGCCCCTGCATCCCCTTGGCTGTGAGGCCCCTGGACAAGCAGA
GGTGGCCAGAGCTGGGAGGCATGGCCCTGGGGTCCCACGAATTTGCTGG
GGAATCTCGTTTTTCTTCTTAAGACTTTTGGGACATGGTTTGACTCCCG
AACATCACCGACGCGTCTCCTGTTTTTCTGGGTGGCCTCGGGACACCTG
CCCTGCCCCCACGAGGGTCAGGACTGTGACTCTTTTTAGGGCCAGGCAG
GTGCCTGGACATTTGCCTTGCTGGACGGGGACTGGGGATGTGGGAGGGA
GCAGACAGGAGGAATCATGTCAGGCCTGTGTGTGAAAGGAAGCTCCACT
GTCACCCTCCACCTCTTCACCCCCCACTCACCAGTGTCCCCTCCACTGT
CACATTGTAACTGAACTTCAGGATAATAAAGTGTTTGCCTCCA
(Genbank Acc. No.NM_000759.4)

### Example 4. Pharmaceutical Dosage Forms

The following formulations illustrate representative pharmaceutical dosage forms that may be used for the therapeutic or prophylactic administration of a composition described herein, or a composition specifically disclosed herein (hereinafter referred to as ‘Composition X’ wherein the composition X comprises a PARP-effector molecule conjugate as described herein):

| (i) Tablet 1 | mg/tablet |
|---|---|
| ‘Composition X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Composition X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Composition X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/mL) | mg/mL |
|---|---|
| ‘Composition X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |

| (v) Injection 2 (10 mg/mL) | mg/mL |
|---|---|
| ‘Composition X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |

| (vi) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

| (vii) Topical Gel 1 | wt. % |
|---|---|
| ‘Composition X’ | 5% |
| Carbomer 934 | 1.25% |
| Triethanolamine | q.s. |
| (pH adjustment to 5-7) | |
| Methyl paraben | 0.2% |
| Purified water | q.s. to 100 g |

| (viii) Topical Gel 2 | wt. % |
|---|---|
| ‘Composition X’ | 5% |
| Methylcellulose | 2% |
| Methyl paraben | 0.2% |
| Propyl paraben | 0.02% |
| Purified water | q.s. to 100 g |

| (ix) Topical Ointment | wt. % |
|---|---|
| ‘Composition X’ | 5% |
| Propylene glycol | 1% |
| Anhydrous ointment base | 40% |
| Polysorbate 80 | 2% |
| Methyl paraben | 0.2% |
| Purified water | q.s. to 100 g |

| (x) Topical Cream 1 | wt. % |
|---|---|
| ‘Composition X’ | 5% |
| White bees wax | 10% |
| Liquid paraffin | 30% |
| Benzyl alcohol | 5% |
| Purified water | q.s. to 100 g |

| (xi) Topical Cream 2 | wt. % |
|---|---|
| ‘Composition X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, and in particular, Cheng et al., Biomacromolecules 2022, 23, 5267-5272; Zhang et al., Chem Sci 2022, 13 (7), 1982-1991; Zhang et al., Nature communications 2019, 10 (1), 4196; Shi et al., Chemical Science 2020, 11 (34), 9303-9308; and U.S. Pat. No. 11,497,787 to Zhang. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A poly ADP-ribose polymerase (PARP)-effector molecule conjugate comprising:
an automodified PARP comprising a plurality of poly ADP-ribose (ADPr) polymers, wherein the plurality of poly ADPr polymers comprise a plurality of 3 ′-azido NAD⁺ moieties; and
an effector molecule conjugated to one or more of the plurality of 3′-azido NAD⁺ moieties of the poly ADPr polymers, wherein at least one of the plurality of 3′-azido NAD⁺ moieties is not conjugated to an effector molecule.

2. The conjugate of claim 1, wherein the plurality of 3′-azido NAD⁺ moieties have a structure according to Formula I: 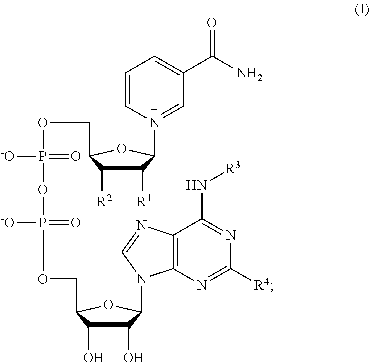
wherein R¹ is OH, R² is N₃, R³ is H, and R⁴ is H.

3. The conjugate of claim 1 wherein the automodified PARP is linked to the effector molecule through an alkyne-derived linking group, a polyethylene glycol linking group, or a combination thereof.

4. The conjugate of claim 3 wherein the alkyne-derived linking group is a linking group derived from an alkyne-PEG₄-NHS ester.

5. The conjugate of claim 1 wherein the automodified PARP is linked to the effector molecule through an alkyne-derived linking group, a cyclooctyne-derived linking group, a polyethylene glycol linking group, or a combination thereof.

6. The conjugate of claim 1 wherein the PARP is selected from the group consisting of PARP1, PARP2, PARP5α, and PARP5β.

7. The conjugate of claim 1 wherein the PARP is PARP1.

8. The conjugate of claim 1 wherein the effector molecule comprises an interleukin, an interferon, a tumor necrosis factor, a granulocyte colony stimulating factor (GCSF), or a granulocyte-macrophage colony stimulating factor (GM-CSF).

9. The conjugate of claim 8 wherein the effector molecule comprises granulocyte colony stimulating factor (GCSF).

10. The conjugate of claim 9 wherein the GCSF comprises SEQ ID NO: 1.

11. The conjugate of claim 1 wherein a ratio of the effector molecule conjugated to the automodified PARP is about 5:1 to about 100:1.

12. A composition comprising the conjugate of claim 1 and a pharmaceutically acceptable carrier.

13. A poly ADP-ribose polymerase (PARP)-granulocyte colony stimulating factor (GCSF) conjugate comprising:
an automodified PARP1 or PARP2 comprising a plurality of poly ADP-ribose (ADPr) polymers on a surface of the automodified PARP1 or PARP2, wherein the poly ADPr polymers comprise a plurality of 3′-azido NAD⁺ moieties according to Formula I: 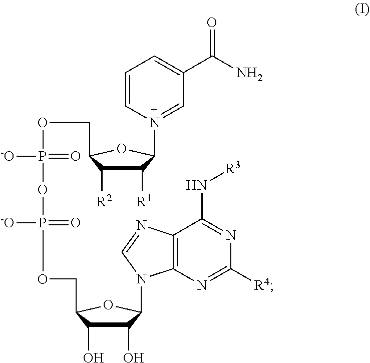
wherein R¹ is OH, R² is N₃, R³ is H, and R⁴ is H; and
GCSF conjugated to one or more of the plurality of 3′-azido NAD⁺ moieties of the poly ADPr polymers, wherein a ratio of the GCSF conjugated to the PARP1 or PARP2 is about 5:1 to about 50:1,
wherein at least one of the plurality of 3′-azido NAD⁺ moieties remain unconjugated.

14. A method of preparing a poly ADP-ribose polymerase (PARP)-effector molecule conjugate comprising:
combining a linker and an effector molecule to provide an effector molecule-linker conjugate;
combining a PARP and a plurality of an azido substituted dinucleotide to provide an automodified PARP comprising a plurality of poly ADP-ribose (ADPr) groups on a surface of the automodified PARP; and
combining the automodified PARP and the effector molecule-linker conjugate under suitable conditions such that the effector molecule-linker conjugate is conjugated to the substituted dinucleotide through click chemistry to form the PARP-effector molecule conjugate.

15. The method of claim 14 wherein the azido substituted dinucleotide is 3′-azido NAD⁺ having a structure according to Formula I: 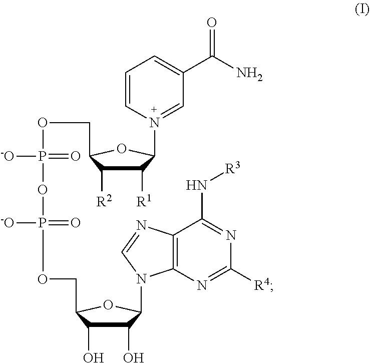
wherein R¹ is OH, R² is N₃, R³ is H, and R⁴ is H.

16. The method of claim 14 wherein the PARP is selected from the group consisting of PARP1, PARP2, PARP5α, or PARP5β.

17. The method of claim 14 wherein a ratio of effector molecule conjugated to the automodified PARP is about 5:1 to about 100:1.

18. A method of stimulating neutrophil cell proliferation comprising:
contacting a neutrophil cell with an effective amount of the conjugate of claim 1, thereby causing proliferation of the neutrophil cell.


SEQUENCE LISTING (6 sequences)

SEQ ID NO: 1 - protein, 207 aa, Homo sapiens
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - DNA, 1513 bp, Homo sapiens
  source 1..1513: mol_type = other DNA
agcccggagcctgcagcccagccccacccagacccatggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagccctgagccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctcca

SEQ ID NO: 3 - DNA, 42 bp, synthetic construct
  source 1..42: mol_type = other DNA
tggtgctcgagccacagggaggtcttaaaattgaatttcagt

SEQ ID NO: 4 - DNA, 66 bp, synthetic construct
  source 1..66: mol_type = other DNA
ccctctagaaataattttgtttaactttaagaaggagatataccatggcggagtcttcggataagc

SEQ ID NO: 5 - DNA, 30 bp, synthetic construct
  source 1..30: mol_type = other DNA
gaattcgacaccactgggacctgcaagttc

SEQ ID NO: 6 - DNA, 25 bp, synthetic construct
  source 1..25: mol_type = other DNA
ccagctagcacctatcaatggtggt